(19) United States
(12) Patent Application Publication
(10) Pub. No.: US 2022/0339234 A1
(43) Pub. Date: Oct. 27, 2022

(54) NATURAL-SUBSTANCE COMPOSITION FOR THE TOPICAL TREATMENT AND CARE OF PSORIATIC SKIN AND OTHER SKIN DISEASES

(71) Applicant: Maria TSAKOURIDOU, Stuttgart (DE)

(72) Inventor: Dimitrios TSAKOURIDIS, Stuttgart (DE)

(73) Assignee: Maria TSAKOURIDOU, Stuttgard (DE)

(21) Appl. No.: 17/613,556

(22) PCT Filed: May 22, 2020
(86) PCT No.: PCT/EP2020/064296

§ 371 Date: Nov. 23, 2021

(30) Foreign Application Priority Data
May 23, 2019 (EP) 19176178.2

(51) Int. Cl. A61K 36/738; A61K 36/48; A61K 36/47; A61K 36/63; A61K 36/54; A61K 35/36; A61P 17/06; A61K 9/00; A61K 9/70

(52) U.S. Cl. CPC A61K 36/738; A61K 36/48; A61K 36/47; A61K 36/63; A61K 36/54; A61K 35/36; A61P 17/06; A61K 9/0014; A61K 9/7023

(57) ABSTRACT

The invention relates to a composition for the topical treatment and care of psoriatic skin. It consists exclusively of natural substances and comprises at the most peanut oil, olive oil, castor oil, avocado oil, rose oil, rose hydrolate, lanolin and phytosterol in the form of a mixture of at least peanut oil, olive oil, castor oil and lanolin.

Psoriasis is a chronic, inflammatory, non-infectious skin disease.

In healthy individuals, the epidermis, which normally renews itself in a regular cycle of approximately 26 to 28 days, continuously sheds aged and cornified skin cells during renewal. This renewal is impaired in psoriasis patients, which results in a greatly accelerated renewal reaction of the epidermis that manifests itself in clearly defined, reddened, raised patches of skin (so-called plaques) that may be covered with a scaly, sometimes silvery shining layer of scales. The joints (for example elbows, knees), scalp and genital area are most commonly affected, but skin appendages such as the fingernails or toenails may also exhibit psoriatic damage. Psoriasis is often accompanied by associated diseases such as arthritis or cardiovascular disease.

The occurrence and course of so-called “flare-ups” in psoriasis patients generally depends on a number of factors. Studies have shown that genetic predispositions are partly responsible, but external conditions, such as infections, stress, nutrition, weather conditions or any additional medication that may have been taken, can also have a significant influence. The pathological situation of psoriasis patients is complex and is accompanied by a particularly strong psychological burden. Since the skin, as an externally visible organ of the body, is noticed by other individuals and thus represents an important means of communication, a visible skin disease, such as psoriasis, often leads to unjustified rejection and exclusion. This places a psychological burden on most psoriasis patients. Consistent treatment of the externally visible symptoms is therefore fundamentally required. In addition to the systemic treatment of the patient, various phototherapy or electrotherapy options are known. However, the topical treatment of psoriatic skin is of primary importance.

Known compositions for the topical treatment and care of psoriatic skin contain, for example, cortisone, antiseptically active dithranol (anthralin) (as in DE 32 39 203), cell proliferation-inhibiting vitamin D derivatives such as calcipotriol or calcitriol (as in WO2008/065514, EP2515865 or WO2012/127037), or salicylic acid and urea as keratolytics. However, these agents cause intolerances in many patients and/or, due to their mode of action, should only be used after medical consultation and are limited to local application to the affected areas.

Products containing the aforementioned substances often only alleviate symptoms temporarily. Psoriasis patients must follow a precise dosage for these treatments and can only apply the products to the affected areas. For example, treatment with cortisone-containing agents can cause the skin to become thin and fragile, which is a major disadvantage especially for patients already affected by psoriasis. Products containing dithranol may cause discolouration, reddening of the skin and additional itching of the skin, and thus often need to be washed off after a certain application time. Products containing coal tar increase the skin's sensitivity to UV rays and are suspected of being carcinogenic.

WO2011/016812A1 discloses a composition for the treatment of psoriasis comprising a) a mixture of oils selected from chia oil, cod liver oil, castor oil, peanut oil and olive oil, b) pure wax and lanolin, c) metallic iodine, camphor and benzoic acid as antiseptics, d) chlorophyll and/or herbal extracts, and e) a pharmaceutically acceptable excipient. This composition contains synthetic components and is therefore not a composition of natural substances that contains only naturally occurring plant or animal substances that are unprocessed or are obtained solely by mechanical or water-based extraction methods. Metallic iodine is a greyish black, shiny metallic solid at room temperature that causes skin discolouration and can lead to allergisation. Camphor can also cause discolouration, reddening of the skin and additional itching of the skin, and thus this composition is associated with side effects.

Psoriasis patients are therefore often skeptical of such active substances and look for alternatives that are easier to use during long-term treatment and do not have any of the aforementioned side effects. They are particularly mindful and critical of the ingredients of products for the care and treatment of psoriatic skin. In particular patients from risk groups requiring particular caution when it comes to medication, such as children or pregnant women, require alternative treatment options.

For some time now, experts in the field have therefore started to manufacture products for the care and treatment of psoriatic skin that are based on natural substances. Natural substances include all chemical compounds that are formed by organisms to fulfil a biological function. Due to their often high structural diversity, natural substances are difficult to produce fully synthetically and are thus usually obtained as extracts from plant or animal components. The biomolecules contained in natural substances include biologically active substances that play an important role in particular in pharmaceutics.

For example, EP 3 013 423 B1 discloses a composition that is based on oils and waxes, with an oily nettle extract (Oleum coctus Urticae). The product is also used inter alia for the treatment of skin with psoriasis.

EP 2 489 358 B1 mentions the natural substance indirubin from Indigo naturalis, also obtained by oil extraction, for the treatment of psoriasis. Vegetable oils are also preferably used here.

EP 1 267 900 B1 also mentions a natural substance, betulin, which essentially comprises triterpenes and is processed in the form of an emulsion for application to psoriatic skin.

Even though the aforementioned natural-substance-based products have higher patient acceptance than conventional medicaments for the treatment of psoriatic skin, they still have disadvantages as regards the production and application thereof.

The cited natural substances are each extracted in complex processes and then processed into specific dosage forms, usually skin oils or emulsions. In some cases, solvents have to be used, such as in the case of the active substance betulin. The processes for producing the products are therefore complex and expensive.

Even though they are natural substances, undesirable side effects cannot always be ruled out. For example, intolerances or overreactions to nettles are known. Even though methods for the production of products containing indirubin that reduce staining of the skin by indirubin are already known, the substance still stains the skin. Hence, patients who are already psychologically burdened owing to the visible skin disease also shy away from this type of product.

The object of the present invention is therefore to provide a composition for the treatment and care of psoriatic skin that is made of natural substances, also referred to as a natural-substance composition, which can be applied to the skin with little risk for as many affected patients as possible, is well tolerated due to a lack of side effects, leads to a complete healing of psoriatic skin, and can nevertheless be produced at low cost and with a low production effort.

The object of the invention is solved by a composition made of natural substances (or natural-substance composition) according to claim 1, which comprises peanut oil (Arachis hypogaea), olive oil (Oleum olivarum), castor oil (Ricinus communis), lanolin and optionally one or more representatives selected from the group consisting of avocado oil (Persea gratissima), rose oil (Rosa damascena), rose hydrolate and phytosterol, or consists (essentially) of said components.

According to the invention, a “composition of natural substances” comprises or consists (essentially) exclusively of naturally occurring plant or animal substances that are unprocessed or are obtained solely by mechanical or water-based extraction methods (flotation, extraction with water, steam distillation, etc.). The mixture of at least peanut oil, olive oil, castor oil and lanolin, and optionally rose oil and/or rose hydrolate and/or lanolin and/or phytosterol, that is contained in the composition according to the invention accordingly comprises or consists (essentially) of substances obtained exclusively in the aforementioned manner. The composition according to the invention is therefore entirely of natural origin, i.e. it does not contain any components that are of synthetic or artificial origin.

The composition according to the invention consists predominantly of vegetable oils, which, in the mixture with lanolin, show a highly beneficial effect on psoriatic skin or eczema, fresh scars, stretch marks, dry skin, skin rashes and skin damaged by itching or sunburn. The cornifications come away and the inflamed, reddened skin calms down. The mixture according to the invention is particularly well tolerated, shows no side effects and is therefore safe for external use by all patient groups. Used regularly, i.e. two or three times a day, for instance, the clinical picture improves significantly. The inflammation of the skin in particular decreases substantially. The composition according to the invention can also lead to complete healing of psoriatic skin when used regularly. Application two to three times a day for a period of at least one to three months is recommended for complete healing.

Psoriasis patients or patients with eczema, fresh scars, stretch marks, dry skin, itching, skin rashes and sunburn, who belong to particular risk groups, such as children and pregnant women, can also safely use the composition according to the invention.

The composition according to the invention has the advantage that it does not discolour the skin, as is the case with other agents known in psoriasis treatment, such as agents containing indigo or iodine (see WO2011/016812A1).

The use of a small number of ingredients in the composition according to the invention has the advantage that the patient can obtain a quick overview of the ingredients. This also greatly limits the risk of allergies.

Finally, production of the composition according to the invention is possible using a simple device. The production costs are also low.

The composition according to the invention preferably comprises or consists (essentially) of peanut oil, olive oil, castor oil and lanolin. The composition according to the invention preferably comprises or consists (essentially) of peanut oil, olive oil, castor oil and lanolin in the concentrations of peanut oil 50 to 60 vol. %, olive oil 25 to 35 vol. %, castor oil 8 to 14 vol. %, and lanolin 1 to 4 vol. %. The concentrations actually selected add up to 100 vol. % (percent by volume) in total.

“Peanut oil” is a vegetable oil pressed from the seeds of peanuts (Arachis hypogaea). Due to the peanut oil, the composition according to the invention is not suitable for peanut allergy sufferers.

“Olive oil”, also called tree oil or, in technical terms, Oleum olivarum, is a vegetable oil extracted from the flesh and pit of olives, the fruit of the olive tree (Olea europaea).

“Castor oil” is a vegetable oil extracted from the seeds of the miracle tree (Ricinus communis) and other members of the Euphorbisceae family, the episperm being removed from the seeds.

“Lanolin”, also called wool wax, is the secretion from the sebaceous glands of sheep, which is extracted from sheep wool.

The composition according to the invention may also comprise peanut oil, olive oil, castor oil, lanolin and rose oil or consist (essentially) thereof. The composition according to the invention may preferably comprise or consist (essentially) of peanut oil, olive oil, castor oil, lanolin and rose oil, each in the following concentrations: peanut oil 50 to 60 vol. %, olive oil 25 to 35 vol. %, castor oil 8 to 14 vol. %, lanolin 1 to 3 vol. %, and rose oil 0.001 to 0.020 vol. %, with the selected concentrations adding up to 100 vol. % in total.

“Rose oil” is extracted from the petals of roses (usually Rosa centifolia or Rosa damascena) by means of steam distillation. The rose oil, together with the other natural substances of the composition according to the invention, has a particularly positive effect on the affected areas of the skin.

According to a further embodiment, the composition according to the invention may also comprise peanut oil, olive oil, castor oil, lanolin and rose hydrolate or consist (essentially) thereof, the concentrations preferably being selected as follows: peanut oil 50 to 60 vol. %, olive oil 25 to 33 vol. %, castor oil 8 to 14 vol. %, lanolin 1 to 4 vol. %, and rose hydrolate 1 to 3 vol. %, adding up to 100 vol. % in total.

“Rose hydrolate” or rose water is produced as a by-product during the distillation of rose oil from rose petals. It contains a particularly high proportion of 2-phenylethanol. Rose hydrolate has an anti-inflammatory and at the same time moisturising effect on the skin.

Another possible embodiment of the composition according to the invention comprises or consists (essentially) of peanut oil, olive oil, castor oil, avocado oil, lanolin and phytosterol, with the following concentrations being preferred: peanut oil 50 to 60 vol. %, castor oil 25 to 35 vol. %, olive oil 5 to 12 vol. %, avocado oil 2 to 8 vol. %, lanolin 1 to 4 vol. %, and phytosterol 0.2 to 0.5 vol. %, the concentrations of the individual substances adding up to 100 vol. % in total.

Avocado oil is a vegetable oil extracted from the fruit of the avocado tree (Persea americana). Like olive oil, it is a pulp oil.

Phytosterols are secondary plant substances comprising at least 70% beta-sitosterol. According to the invention, plant phytosterol is preferably used, which is preferably present as a white solid. Pine phytosterol (Pinus SSP), such as the phytosterol with CAS No. 949109-75-5 (PHYTOPIN®), is preferably used. This phytosterol comprises 70 to 80% of beta-sitosterol, a maximum of 15% sitostanol, a maximum of 6 to 10% campesterol 6-10, a maximum of 2% campestanol and a maximum of 3% other sterols.

The composition according to the invention may furthermore also comprise peanut oil, olive oil, castor oil, avocado oil, lanolin, rose oil and phytosterol or consist (essentially) thereof, with the following concentrations being preferred: peanut oil 50 to 60 vol. %, castor oil 25 to 35 vol. %, olive oil 3 to 7 vol. %, avocado oil 4 to 8 vol. %, lanolin 2 to 4 vol. %, rose oil 0.01 to 0.02 vol. %, and phytosterol 0.2 to 0.5 vol. %, with the selected concentrations adding up to 100 vol. % in total.

According to the invention, the castor oil and/or the olive oil may furthermore be in ozonated form. The oxygen-enriched oils have the effect of increasing oxygen supply to the skin, which accelerates healing of the affected areas.

According to the invention, the composition according to the invention may also be a component of a body care product, in particular a lotion, a cream, an ointment, a wash lotion or a shampoo, as an ingredient effective solely for the treatment of psoriatic skin.

It is furthermore provided within the scope of the invention to administer the composition on a carrier material that is intended for application to the psoriatic skin to be treated, the carrier material containing a specific amount of the composition according to the invention for acting on the skin to be treated. In this manner, the composition according to the invention can act on the skin from the medicinal plaster over a longer period of time, i.e. over several hours, which further improves the success of the treatment. The patient's clothing is furthermore advantageously protected since contact with the composition can be avoided.

Five possible embodiments of the invention are provided below:

TABLE 1
Embodiment 1
Embodiment I

| Ingredients | Concentration vol. % |
|---|---|
| Peanut oil (Arachis hypogaea) | 55.00 |
| Castor oil (Ricinus communis) | 30.00 |
| Olive oil (Oleum olivarum) | 6.50 |
| Avocado oil (Persea gratissima) | 6.00 |
| Lanolin | 2.25 |
| Phytosterol (plant) | 0.25 |

TABLE 2
Embodiment 2
Embodiment II

| Ingredients | Concentration vol. % |
|---|---|
| Peanut oil (Arachis hypogaea) | 55.98 |
| Castor oil (Ricinus communis) | 30.00 |
| Olive oil (Oleum olivarum) | 5.00 |
| Avocado oil (Persea gratissima) | 5.75 |
| Lanolin | 3.00 |
| Rose oil (Rosa damascena) | 0.02 |
| Phytosterol (plant) | 0.25 |

TABLE 3
Embodiment 3
Embodiment III

| Ingredients | Concentration vol. % |
|---|---|
| Peanut oil (Arachis hypogaea) | 55.00 |
| Ozonated castor oil (Ricinus communis) | 30.00 |
| Ozonated olive oil (Oleum europaea) | 10.00 |
| Lanolin | 2.40 |
| Avocado oil (Persea gratissima) | 2.35 |
| Phytosterol (plant) | 0.25 |

TABLE 4
Embodiment 4
Embodiment IV

| Ingredients | Concentration vol. % |
|---|---|
| Peanut oil (Arachis hypogaea) | 55.98 |
| Olive oil (Oleum olivarum) | 30.00 |
| Castor oil (Ricinus communis) | 12.00 |
| Lanolin | 2.00 |
| Rose oil (Rosa damascena) | 0.02 |

TABLE 5
Embodiment 5
Embodiment V

| Ingredients | Concentration vol. % |
|---|---|
| Peanut oil (Arachis hypogaea) | 55.0 |
| Olive oil (Oleum olivarum) | 28.00 |
| Castor oil (Ricinus communis) | 10.5 |
| Lanolin | 3.0 |
| Rose water | 1.5 |

TABLE 6
Embodiment 6
Embodiment VI

| Ingredients | Concentration vol. % |
|---|---|
| Peanut oil (Arachis hypogaea) | 54.98 |
| Ozonated castor oil (Ricinus communis) | 30.00 |
| Ozonated olive oil (Oleum europaea) | 10.00 |
| Lanolin | 2.40 |
| Avocado oil (Persea gratissima) | 2.35 |
| Phytosterol (plant) | 0.25 |
| Rose oil (Rosa damascena) | 0.02 |

The natural-substance composition according to the invention can be stored at room temperature for 20 to 24 months. The natural-substance composition according to the invention is therefore stable at room temperature for 20 to 24 months.

The natural-substance composition according to the invention may preferably be in the form of an oil, for example a pale yellow oil. This has the advantage that it easily penetrates into the skin to be treated and preferably does not lead to discolouration of the skin. The natural-substance composition according to the invention may alternatively also be in the form of a cream, a gel, a lotion or a paste. It is particularly preferably in the form of an oil.

The aforementioned compositions can advantageously be produced with little effort. The individual oils are mixed together one by one and heated at 56 to 58° C. Lanolin and optionally phytosterol are then added and dissolved. Alternatively, the peanut oil is optionally mixed together with the phytosterol and heated to 50 to a maximum of 75° C. Olive oil, castor oil and optionally one or more representatives selected from the group consisting of avocado oil, rose oil and rose hydrolate are subsequently added together with lanolin, and the obtained mixture is cooled.

The effect of the compositions according to the invention in the aforementioned embodiments has already been successfully tested on several patients. The subjects were all affected by psoriasis type I. In a preferred embodiment, the plaques had significantly decreased in all patients within 8 to 10 weeks of consistent treatment with the composition according to the invention, and in some cases had also healed completely. According to the invention, a “consistent treatment” involves the patient applying a thin layer (i.e. a few drops) of the composition according to the invention to the affected area at least twice a day (for example in the morning and the evening), rubbing it in, and waiting until it is completely absorbed, which may take approximately 15 to 20 min. A thin layer of the composition according to the invention may also be applied to the affected area three times a day (for example in the morning, afternoon and evening). This application can in particular immediately relieve itching.

The effect of the composition according to the invention is explained below by means of examples.

### EXAMPLE 1

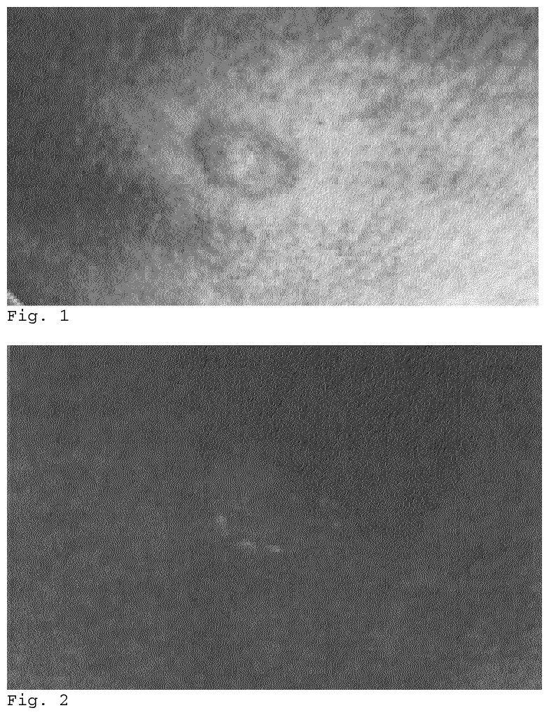

The patient, 19 years old, is affected by psoriasis type I on the arm and thigh, and by psoriasis capitis on the scalp. The plaques on the thigh and arm were treated with the composition according to the invention (Embodiment 4). The test treatment started on 15 Jan. 2018 and ended on 29 Mar. 2018. The composition was applied to the affected areas in the morning and evening. The areas were covered with a plaster to allow the composition to act on the skin even under clothing. FIGS. 1 to 5 show the course of healing of the affected areas of skin on the thigh.

These show:

FIG. 1 the treated area of skin on day 1 of treatment,

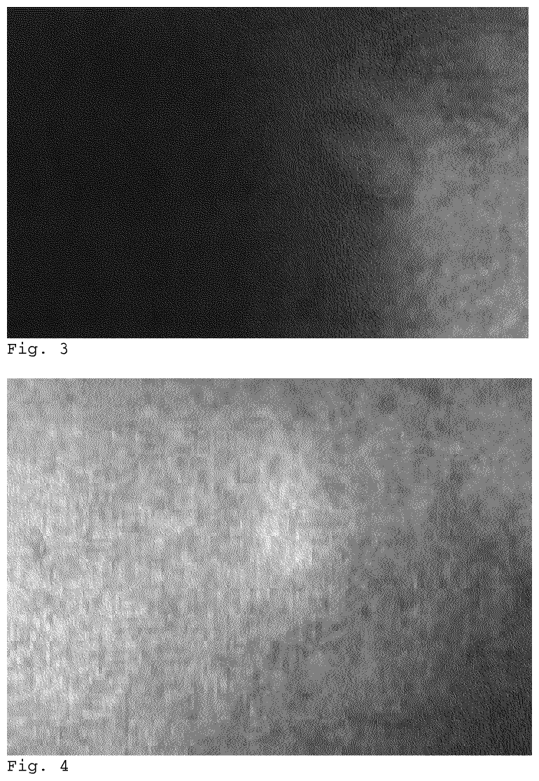

FIG. 2 the treated area of skin on day 30 of treatment,

FIG. 3 the treated area of skin on day 62 of treatment,

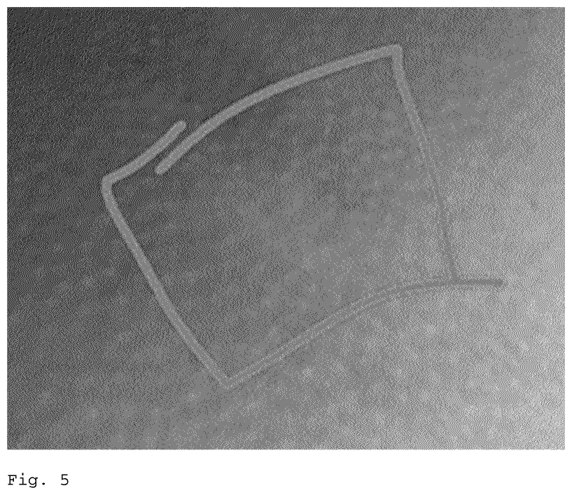

FIG. 4 the treated area of skin on day 72 of treatment, and

FIG. 5 the treated area of skin on day 75 of treatment.

### EXAMPLE 2

A monocentric, clinical-dermatological application study with 20 subjects was carried out with the composition according to embodiment II of the invention and with twice-daily application to acute eczema caused by psoriasis over a period of 8 weeks.

The primary study objectives were to test skin tolerance (all skin reactions and possible other side effects were recorded in the reaction form from the start of the study until the end of the study and 30 days beyond). Secondary study objectives were to test effectiveness by means of a dermatological assessment (measurement time points: T_{4 weeks} and T_{8 weeks}).

The inclusion criteria were: 18 years or older, male and female subjects with healthy skin, skin type: any, subjects with acute eczema caused by psoriasis (not requiring medical treatment), written consent of subjects or legal guardians was provided.

The exclusion criteria were: severe or chronic skin inflammation, severe internal or chronic diseases, use of medicaments that could affect skin reactions (glucocorticoids, antiallergics, topical immunomodulators, etc.), application of preparations and care products containing active substances 7 to 10 days prior to the start of the test, severe allergies or serious side effects from cosmetic preparations, sunbathing or solarium visits during the study, known cancer, and pregnancy/breastfeeding.

### Dermatological Test Results:

The tests were performed according to clinical-dermatological assessment criteria. 20 test persons showed healthy skin in the test area before, during and after the application study. No pathological skin changes were observed in any form. No interruptions to the test or even treatments by a dermatologist occurred at all. The composition according to the invention was very well tolerated by these subjects and did not lead to any dermatologically relevant skin changes.

After 14 days of use, one subject reported a painful burning sensation on both elbows after application to the skin affected by plaque psoriasis. Upon examination, the area of the skin was also somewhat irritated and slightly red. The skin of the acute eczema caused by psoriasis was intact upon examination. The subject did not have a nut allergy. The subject discontinued the study. The symptoms affecting the eczema caused by psoriasis subsided completely after about four days, no medical treatment was required.

### Dermatological Assessment:

TABLE 6
Intensities of the parameter redness in the
test area before testing and after 8 weeks

| Subject no. | Tₒ | T_{8 weeks} | Difference | Change [%] |
|---|---|---|---|---|
| 1 | 8.0 | 7.0 | −1.0 | −12.5 |
| 2 | 10.0 | 10.0 | 0.0 | 0.0 |
| 3 | 6.0 | 2.0 | −4.0 | −66.7 |
| 4 | 7.0 | 7.0 | 0.0 | 0.0 |
| 5 | 4.0 | n. d. | n. d. | n.d. |
| 6 | 8.0 | 5.0 | −3.0 | −37.5 |
| 7 | 10.0 | 8.0 | −2.0 | −20.0 |
| 8 | 9.0 | 5.0 | −4.0 | −44.4 |
| 9 | 4.0 | 4.0 | 0.0 | 0.0 |
| 10 | 6.0 | 7.0 | 1.0 | 16.7 |
| 11 | 6.0 | 6.0 | 0.0 | 0.0 |
| 12 | 5.0 | 4.0 | −1.0 | −20.0 |
| 13 | 7.0 | 4.0 | −3.0 | −42.9 |
| 14 | 8.0 | 7.0 | −1.0 | −12.5 |
| 15 | 4.0 | 3.0 | −1.0 | −25.0 |
| 16 | 6.0 | 8.0 | 2.0 | 33.3 |
| 17 | 8.0 | 5.0 | −3.0 | −37.5 |
| 18 | 8.0 | 0.0 | −8.0 | −100.0 |
| 19 | 8.0 | 6.0 | −2.0 | −25.0 |
| 20 | 8.0 | 7.0 | −1.0 | −12.5 |
| 21 | 8.0 | 6.0 | −2.0 | −25.0 |
| Average | 7.0 | 5.6 | −1.4 | −20.0 |
| Minimum | 4.0 | 0.0 | −8.0 | −100.0 |
| Maximum | 10.0 | 10.0 | 2.0 | 33.3 |
| Stand. dev. | 1.8 | 2.3 | 2.2 | 29.3 |

TABLE 7
Intensities of the parameter scales in the
test area before testing and after 8 weeks

| Subject no. | Tₒ | T_{8 weeks} | Difference | Change [%] |
|---|---|---|---|---|
| 1 | 8.0 | 4.0 | −4.0 | −50.0 |
| 2 | 5.0 | 3.0 | −2.0 | −40.0 |
| 3 | 5.0 | 2.0 | −3.0 | −60.0 |
| 4 | 8.0 | 5.0 | −3.0 | −37.5 |
| 5 | 9.0 | n. d. | n. d. | n.d. |
| 6 | 4.0 | 2.0 | −2.0 | −50.0 |
| 7 | 10.0 | 6.0 | −4.0 | −40.0 |
| 8 | 7.0 | 2.0 | −5.0 | −71.4 |
| 9 | 7.0 | 3.0 | −4.0 | −57.1 |
| 10 | 7.0 | 3.0 | −4.0 | −57.1 |
| 11 | 6.0 | 4.0 | −2.0 | −33.3 |
| 12 | 7.0 | 3.0 | −4.0 | −57.1 |
| 13 | 3.0 | 1.0 | −2.0 | −66.7 |
| 14 | 5.0 | 2.0 | −3.0 | −60.0 |
| 15 | 4.0 | 2.0 | −2.0 | −50.0 |
| 16 | 10.0 | 10.0 | 0.0 | 0.0 |
| 17 | 6.0 | 3.0 | −3.0 | −50.0 |
| 18 | 6.0 | 1.0 | −5.0 | −83.3 |
| 19 | 7.0 | 5.0 | −2.0 | −28.6 |
| 20 | 3.0 | 3.0 | 0.0 | 0.0 |
| 21 | 8.0 | 7.0 | −1.0 | −12.5 |
| Average | 6.4 | 3.6 | −2.8 | −43.8 |
| Minimum | 3.0 | 1.0 | −5.0 | −83.3 |
| Maximum | 10.0 | 10.0 | 0.0 | 0.0 |
| Stand. dev. | 2.0 | 2.2 | 1.4 | 22.1 |

TABLE 8
Intensities of the parameter lichenification (increase,
thickening of the skin in the area of acute eczema)
in the test area before testing and after 8 weeks

| Subject no. | Tₒ | T_{8 weeks} | Difference | Change [%] |
|---|---|---|---|---|
| 1 | 10.0 | 8.0 | −2.0 | −20.0 |
| 2 | 10.0 | 8.0 | −2.0 | −20.0 |
| 3 | 6.0 | 3.0 | −3.0 | −50.0 |
| 4 | 8.0 | 6.0 | −2.0 | −25.0 |
| 5 | 7.0 | n.d. | n.d. | n.d. |
| 6 | 7.0 | 4.0 | −3.0 | −42.9 |
| 7 | 10.0 | 5.0 | −5.0 | −50.0 |
| 8 | 7.0 | 3.0 | −4.0 | −57.1 |
| 9 | 6.0 | 4.0 | −2.0 | −33.3 |
| 10 | 6.0 | 4.0 | −2.0 | −33.3 |
| 11 | 5.0 | 5.0 | 0.0 | 0.0 |
| 12 | 6.0 | 4.0 | −2.0 | −33.3 |
| 13 | 3.0 | 1.0 | −2.0 | −66.7 |
| 14 | 4.0 | 2.0 | −2.0 | −50.0 |
| 15 | 7.0 | 2.0 | −5.0 | −71.4 |
| 16 | 10.0 | 10.0 | 0.0 | 0.0 |
| 17 | 6.0 | 4.0 | −2.0 | −33.3 |
| 18 | 7.0 | 1.0 | −6.0 | −85.7 |
| 19 | 2.0 | 2.0 | 0.0 | 0.0 |
| 20 | 7.0 | 6.0 | −1.0 | −14.3 |
| | 8.0 | 5.0 | −3.0 | −37.5 |
| Average | 6.8 | 4.4 | −2.4 | −36.2 |
| Minimum | 2.0 | 1.0 | −6.0 | −85.7 |
| Maximum | 10.0 | 10.0 | 0.0 | 0.0 |
| Stand. dev. | 2.2 | 2.4 | 1.6 | 23.8 |

### Assessment of the Study Results:

### Skin Tolerance:

20 subjects applied the composition according to the invention twice a day over a period of eight weeks primarily to the acute eczema caused by psoriasis. From a clinical-dermatological point of view, no relevant skin reactions occurred in the test area, and the composition according to the invention was very well tolerated. Neither intolerance reactions in the sense of irritation nor allergic reactions (contact dermatitis) were observed. After 14 days of use, one subject reported a painful burning sensation on both elbows after application to the skin affected by plaque psoriasis. Upon examination, the area of the skin was also somewhat irritated and slightly red. The subject did not have a nut allergy. The skin of the acute eczema caused by psoriasis was intact upon examination. The symptoms affecting the eczema caused by psoriasis subsided completely after about four days, no medical treatment was required.

### Effectiveness:

The effectiveness of the composition according to the invention in respect of the psoriasis symptoms of redness, scales and lichenification (increase of the skin in the area of acute eczema) was determined by means of a dermatological assessment. A reduction in redness of −20.0%, in scales of −43.8%, and in lichenification (increase of the skin in the area of acute eczema) of −36.2% was shown in the test area.

1. A composition of natural substances for the topical treatment and care of psoriatic skin, consisting essentially of peanut oil, olive oil, castor oil, avocado oil, rose oil, rose hydrolate, lanolin and phytosterol in the form of a mixture of at least peanut oil, olive oil, castor oil and lanolin.

2. The composition according to claim 1, characterised in that it consists essentially of
(i) peanut oil, olive oil, castor oil and lanolin, the composition comprising peanut oil, olive oil, castor oil and lanolin in the following concentrations:
peanut oil 50 to 60 vol. %,
olive oil 25 to 35 vol. %,
castor oil 8 to 14 vol. %, and
lanolin 1 to 4 vol. %,
the selected concentrations adding up to 100 vol. % in total; or
(ii) peanut oil, olive oil, castor oil, lanolin and rose oil, the composition comprising peanut oil, olive oil, castor oil, lanolin and rose oil in the following concentrations:
peanut oil 50 to 60 vol. %,
olive oil 25 to 35 vol. %,
castor oil 8 to 14 vol. %,
lanolin 1 to 3 vol. %, and
rose oil 0.001 to 0.020 vol. %,
the selected concentrations adding up to 100 vol. % in total; or
(iii) peanut oil, olive oil, castor oil, lanolin and rose hydrolate, the composition comprising peanut oil, olive oil, castor oil, lanolin and rose hydrolate in the following concentrations:
peanut oil 50 to 60 vol. %,
olive oil 25 to 33 vol. %,
castor oil 8 to 14 vol. %,
lanolin 1 to 4 vol. %, and
rose hydrolate 1 to 3 vol. %,
the selected concentrations adding up to 100 vol. % in total; or
(iv) peanut oil, olive oil, castor oil, avocado oil, lanolin and phytosterol, the composition comprising peanut oil, olive oil, castor oil, avocado oil, lanolin and phytosterol in the following concentrations:
peanut oil 50 to 60 vol. %,
castor oil 25 to 35 vol. %,
olive oil 5 to 12 vol. %,
avocado oil 2 to 8 vol. %,
lanolin 1 to 4 vol. %, and
phytosterol 0.2 to 0.5 vol. %,
the selected concentrations adding up to 100 vol. % in total; or
(v) peanut oil, olive oil, castor oil, avocado oil, lanolin, rose oil and phytosterol, the composition comprising peanut oil, olive oil, castor oil, avocado oil, lanolin, rose oil and phytosterol in the following concentrations:
peanut oil 50 to 60 vol. %,
castor oil 25 to 35 vol. %,
olive oil 3 to 7 vol. %,
avocado oil 4 to 8 vol. %,
lanolin 2 to 4 vol. %,
rose oil 0.001 to 0.020 vol. %, and
phytosterol 0.2 to 0.5 vol. %,
the selected concentrations adding up to 100 vol. % in total.

3. The composition according to claim 1, characterised in that it consists essentially of the following components:

| | |
|---|---|
| Peanut oil | 55.98 vol. % |
| Castor oil | 30.00 vol. % |
| Olive oil | 5.00 vol. % |
| Avocado oil | 5.75 vol. % |
| Lanolin | 3.00 vol. % |
| Rose oil | 0.02 vol. % |
| Phytosterol (plant) | 0.25 vol. % |

4. The composition according to claim 1, characterised in that it contains exclusively naturally occurring plant or animal substances that are unprocessed or are obtained solely by mechanical or water-based extraction methods.

5. The composition according to claim 1, characterised in that the castor oil and/or the olive oil are in ozonated form.

6. The composition according to claim 1, characterised in that it is in the form of an oil, a cream, a gel, a lotion or a paste, preferably in the form of an oil.

7. The composition according to claim 1, characterised in that it is intended for the topical treatment and care of psoriatic skin.

8. The composition according to claim 1 as a component of a body care product, in particular a lotion, a cream, an ointment, a wash lotion or a shampoo, effective solely for the treatment of psoriatic skin.

9. A medicinal plaster comprising a carrier material for application to the psoriatic skin to be treated, wherein the carrier material contains a specific amount of the composition according to claim 1 for acting on the skin to be treated.

10. The composition according to claim 1 for use as a medicament.

11. The composition according to claim 1 for use in the treatment of a skin condition, wherein the skin condition is preferably selected from the group consisting of eczema, psoriasis, fresh scars, stretch marks, dry skin, itching, skin rashes and sunburn.

12. The composition for use according to claim 11, wherein the skin condition is psoriasis.

13. The composition for use according to claim 11, wherein the composition is applied to the skin to be treated twice or three times a day.

14. A method for the production of the composition according to claim 1, comprising the following steps:
(i) Mixing peanut oil and optionally phytosterol,
(ii) Heating the obtained mixture to 50 to 75° C.,
(iii) Adding olive oil, castor oil, and optionally one or more representatives selected from the group consisting of avocado oil, rose oil and rose hydrolate, and dissolving the lanolin, and
(iv) Cooling the obtained mixture.

15. The medicinal plaster according to claim 9 for use as a medicament.

16. The medicinal plaster according to claim 9 for use in the treatment of a skin condition, wherein the skin condition is preferably selected from the group consisting of eczema, psoriasis, fresh scars, stretch marks, dry skin, itching, skin rashes and sunburn.

17. The medicinal plaster for use according to claim 16, wherein the skin condition is psoriasis.

18. The medicinal plaster for use according to claim 16, wherein the medicinal plaster is applied to the skin to be treated twice or three times a day.

19. The medicinal plaster for use according to claim 17, wherein the medicinal plaster is applied to the skin to be treated twice or three times a day.
